(19) United States
(12) Patent Application Publication
(10) Pub. No.: US 2009/0204209 A1
(43) Pub. Date: Aug. 13, 2009

(54) Accommodative Intraocular Lens System

(76) Inventor: Son Trung Tran, Arlington, TX (US)

Correspondence Address: ALCON, IP LEGAL, TB4-8, 6201 SOUTH FREEWAY, FORT WORTH, TX, 76134, US

(21) Appl. No.: 12/420,517

(22) Filed: Apr. 8, 2009

Related U.S. Application Data
(63) Continuation of application No. 11/183,399, filed on Jul. 18, 2005, now abandoned.

(51) Int. Cl. A61F 2/16

(52) U.S. Cl. 623/6.34

(57) ABSTRACT

A two-optic accommodative lens system. The first lens has a negative power and is located posteriorly within the capsular bag and lying against the posterior capsule. The periphery of the first lens is attached to a ring-like structure having sloping sides. The second lens is located anteriorly to the first lens within of the capsular bag and is of a positive power. The peripheral edge of the second lens contains a plurality of haptics that project posteriorly from the second lens and toward the first lens. The haptics are relatively firm, yet still flexible and ride within the sloping sides of the ring-like structure, so that flattening or steepening of the capsule in reaction to movement of the ciliary muscle and corresponding shrinkage of the capsular bag causes the second lens to move along the optical axis of the lens system.

### BACKGROUND OF THE INVENTION

This application claims priority from, and is a continuation of U.S. patent application Ser. No. 11/183,399 filed on Jul. 18, 2005.

This invention relates generally to the field of intraocular lenses (IOL) and, more particularly, to accommodative IOLs.

The human eye in its simplest terms functions to provide vision by transmitting light through a clear outer portion called the cornea, and focusing the image by way of a crystalline lens onto a retina. The quality of the focused image depends on many factors including the size and shape of the eye, and the transparency of the cornea and the lens.

When age or disease causes the lens to become less transparent, vision deteriorates because of the diminished light which can be transmitted to the retina. This deficiency in the lens of the eye is medically known as a cataract. An accepted treatment for this condition is surgical removal of the lens and replacement of the lens function by an artificial intraocular lens (IOL).

In the United States, the majority of cataractous lenses are removed by a surgical technique called phacoemulsification. During this procedure, an opening is made in the anterior capsule and a thin phacoemulsification cutting tip is inserted into the diseased lens and vibrated ultrasonically. The vibrating cutting tip liquifies or emulsifies the lens so that the lens may be aspirated out of the eye. The diseased lens, once removed, is replaced by an artificial lens.

In the natural lens, bifocality of distance and near vision is provided by a mechanism known as accommodation. The natural lens, early in life, is soft and contained within the capsular bag. The bag is suspended from the ciliary muscle by the zonules. Relaxation of the ciliary muscle tightens the zonules, and stretches the capsular bag. As a result, the natural lens tends to flatten. Tightening of the ciliary muscle relaxes the tension on the zonules, allowing the capsular bag and the natural lens to assume a more rounded shape. In the way, the natural lens can be focus alternatively on near and far objects.

As the lens ages, it becomes harder and is less able to change shape in reaction to the tightening of the ciliary muscle. This makes it harder for the lens to focus on near objects, a medical condition known as presbyopia. Presbyopia affects nearly all adults over the age of 45 or 50.

Prior to the present invention, when a cataract or other disease required the removal of the natural lens and replacement with an artificial IOL, the IOL was a monofocal lens, requiring that the patient use a pair of spectacles or contact lenses for near vision. Advanced Medical Optics has been selling a bifocal IOL, the Array lens, for several years, but due to quality of issues, this lens has not been widely accepted.

Several designs for accommodative IOLs are being studied. For example, several designs manufactured by C&C Vision are currently undergoing clinical trials. See U.S. Pat. Nos. 6,197,059, 5,674,282, 5,496,366 and 5,476,514 (Cumming), the entire contents of which being incorporated herein by reference. The lens described in these patents is a single optic lens having flexible haptics that allows the optic to move forward and backward in reaction to movement of the ciliary muscle. A similar designs are described in U.S. Pat. No. 6,302,911 B1 (Hanna), U.S. Pat. Nos. 6,261,321 B1 and 6,241,777 B1 (both to Kellan), the entire contents of which being incorporated herein by reference. The amount of movement of the optic in these single-lens systems, however, may be insufficient to allow for a useful range of accommodation. In addition, as described in U.S. Pat. Nos. 6,197,059, 5,674,282, 5,496,366 and 5,476,514, the eye must be paralyzed for one to two weeks in order for capsular fibrosis to entrap the lens that thereby provide for a rigid association between the lens and the capsular bag. In addition, the commercial models of these lenses are made from a hydrogel or silicone material. Such materials are not inherently resistive to the formation of posterior capsule opacification (“PCO”). The only treatment for PCO is a capsulotomy using a Nd:YAG laser that vaporizes a portion of the posterior capsule. Such destruction of the posterior capsule may destroy the mechanism of accommodation of these lenses.

There have been some attempts to make a two-optic accommodative lens system. For example, U.S. Pat. No. 5,275,623 (Sarfarazi), WIPO Publication No. 00/66037 (Glick, et al.) and WO 01/34067 A1 (Bandhauer, et al), the entire contents of which being incorporated herein by reference, all disclose a two-optic lens system with one optic having a positive power and the other optic having a negative power. The optics are connected by a hinge mechanism that reacts to movement of the ciliary muscle to move the optics closer together or further apart, thereby providing accommodation. In order to provide this “zoom lens” effect, movement of the ciliary muscle must be adequately transmitted to the lens system through the capsular bag, and none of these references disclose a mechanism for ensuring that there is a tight connection between the capsular bag and the lens system. In addition, none of these lenses designs have addressed the problem with PCO noted above.

Therefore, a need continues to exist for a safe and stable accommodative intraocular lens system that provides accommodation over a broad and useful range.

### BRIEF SUMMARY OF THE INVENTION

The present invention improves upon the prior art by providing a two-optic accommodative lens system. The first lens has a negative power and is located posteriorly within the capsular bag and lying against the posterior capsule. The periphery of the first lens is attached to a ring-like structure having sloping sides. The second lens is located anteriorly to the first lens within of the capsular bag and is of a positive power. The peripheral edge of the second lens contains a plurality of haptics that project posteriorly from the second lens and toward the first lens. The haptics are relatively firm, yet still flexible and ride within the sloping sides of the ring-like structure, so that flattening or steepening of the capsule in reaction to movement of the ciliary muscle and corresponding shrinkage of the capsular bag causes the second lens to move along the optical axis of the lens system.

Accordingly, one objective of the present invention is to provide a safe and biocompatible intraocular lens.

Another objective of the present invention is to provide a safe and biocompatible intraocular lens that is easily implanted in the posterior chamber.

Still another objective of the present invention is to provide a safe and biocompatible intraocular lens that is stable in the posterior chamber.

Still another objective of the present invention is to provide a safe and biocompatible accommodative lens system.

These and other advantages and objectives of the present invention will become apparent from the detailed description and claims that follow.

### BRIEF DESCRIPTION OF THE DRAWING

FIG. 1 is an enlarged perspective view of the lens system of the present invention.

FIG. 2 is an enlarged cross-sectional view of the lens system of the present invention.

FIG. 3 is an enlarged top plan view of the first lens of the lens system of the present invention.

FIG. 4 is an enlarged cross-sectional view of the first lens of the lens system of the present invention taken at line 4-4 in FIG. 3.

FIG. 5 is an enlarged detail view taken at circle 5 in FIG. 4.

FIG. 6 is an enlarged top plan view of the second lens of the lens system of the present invention.

FIG. 7 is an enlarged cross-sectional view of the second lens of the lens system of the present invention taken at line 7-7 in FIG. 6.

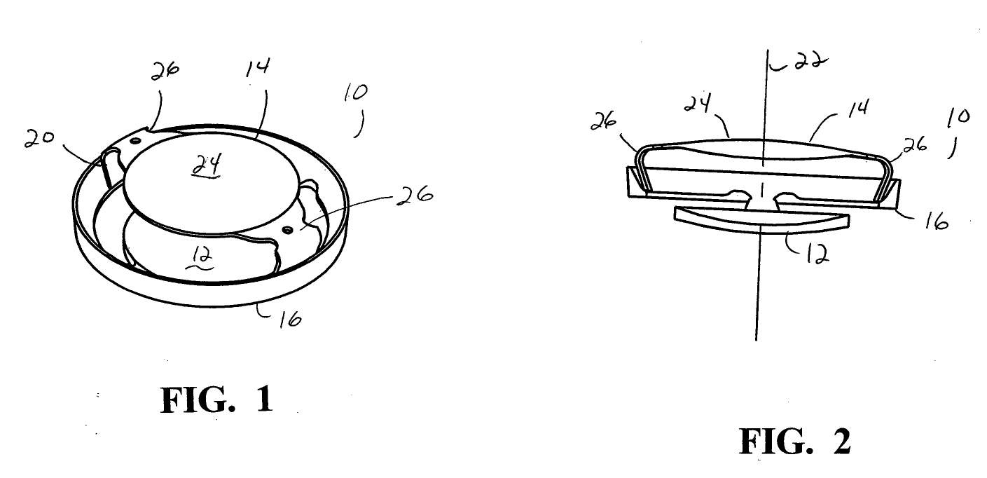

### DETAILED DESCRIPTION OF THE INVENTION

As best seen in the figures, lens system 10 of the present invention generally consists of posterior lens 12, anterior lens 14 and circumferential ring 16. Lens 12 is preferably integrally formed with ring 16 and connected to ring 16 by a plurality of first haptics 18. Lens 12 preferably is made from a soft, foldable material that is inherently resistive to the formation of PCO, such as a soft acrylic. Lens 14 preferable is made from a soft, foldable material such as a hydrogel, silicone or soft acrylic. Lens 12 may be any suitable power, but preferably has a negative power. Lens 14 may also be any suitable power but preferably has a positive power. The relative powers of lenses 12 and 14 should be such that the axial movement of lens 14 toward or away from lens 12 should be sufficient to adjust the overall power of lens system 10 at least one diopter and preferably, at least three to four diopters, calculation of such powers of lenses 12 and 14 being within the capabilities of one skilled in the art of designing ophthalmic lenses by, for example, using the following equations:

P=P₁+P₂−T/n*P₁P₂ (1)

δP=−δT/n*P₁P₂ (2)

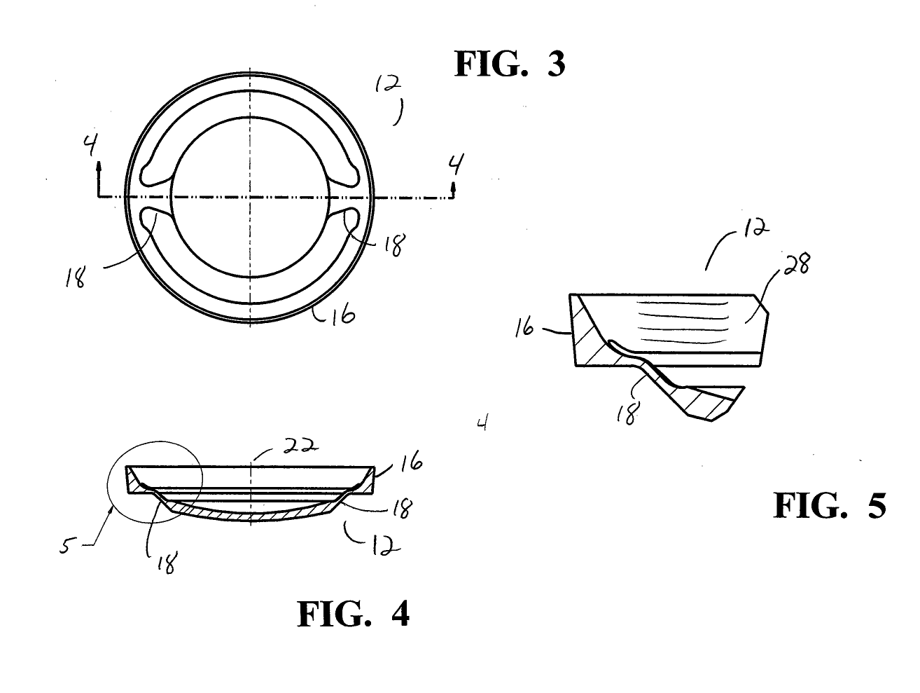

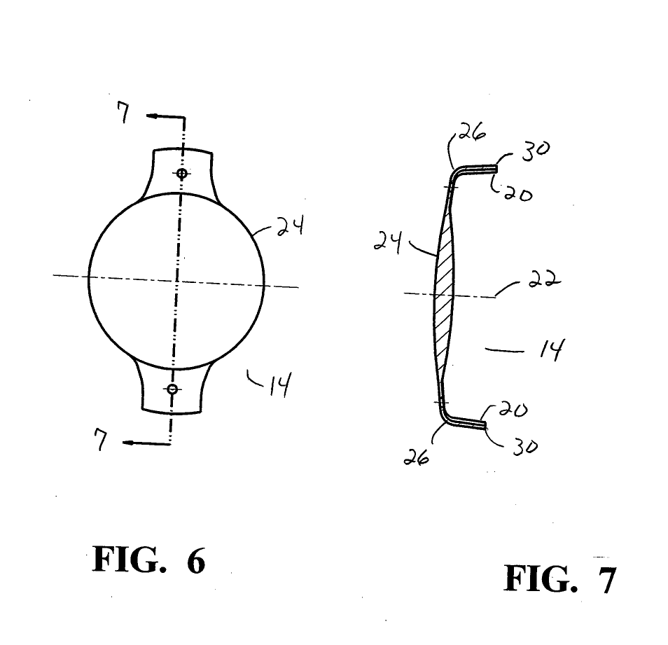

As best seen in FIGS. 3 and 4, lens 12 is generally symmetrical about optical axis 22. First haptics 18 are relatively stiff, so as to allow some, but not excessive, flexing in response to ciliary muscle contraction and relaxation. Interior 28 of ring 16 has generally sloping sides, in other words, the interior diameter of ring 16 increases toward lens 14 along optical axis 22. As best seen in FIGS. 6 and 7, lens 14 contains a plurality of second haptics 20 that project away from optic 24 of lens 14 along optical axis 22. Second haptics 20 are connected to lens 14 by relatively flexible hinge regions 26 that allow second haptics 20 to exhibit resistive, spring-like movement. When compressed, second haptics 20 store energy, releasing that energy when uncompressed.

In use, lens 12 is implanted into the capsular bag prior to the implantation of lens 14. Lens 12 is held within the capsular bag by ring 16 and first haptics 18. Lens 14 is implanted so that second haptics 20 ride within sloped sides of interior 28 of ring 16. Lenses 12 and 14 are free-floating and not connected to each other. Upon implantation of lens 14, lens 14 will flex at hinge regions 26 in response to flattening and steepening of the lens capsule resulting from contraction and relaxation of the ciliary muscles. Flattening of the capsule caused by relaxation of the ciliary will cause tips 30 of second haptics 20 to move toward each other, storing energy in second haptics 20 and hinge regions 26 and allowing tips 30 to slide down the sloped sides of interior 28 and toward lens 12. Such movement will cause lens 14 to move posteriorly along optical axis 22 toward lens 12. When the ciliary muscles contract, the capsule will steepen and the energy stored in second haptics 20 will cause tips 30 to move anteriorly along the sloped sides of interior 28. Such movement will cause lens 14 to move anteriorly along optical axis 22 away from lens 12.

This description is given for purposes of illustration and explanation. It will be apparent to those skilled in the relevant art that changes and modifications may be made to the invention described above without departing from its scope or spirit.

I claim:

1. An intraocular lens system, comprising:
a) a first lens having a first plurality of first haptics;
b) a continuous ring attached to the first lens by the first plurality of haptics, the ring having an interior with generally sloping sides, the first lens, the first plurality of haptics and the ring being integrally formed as a single piece; and
c) a second lens having a plurality of second haptics, the second haptics having tips that slide along the sloping sides of the ring in the direction of an optical axis of the lens system.

2. The lens of claim 1 wherein the second haptics are attached to the second lens by hinge regions that allow the second lens to move away from the first lens.

3. The lens of claim 1 wherein the first lens and the second lens further comprise a soft acrylic material.

4. The lens of claim 1 wherein the second lens further comprises a hydrogel material.

5. The lens of claim 1 wherein the second lens further comprises a silicone material.
